(19) United States
(12) Patent Application Publication
(10) Pub. No.: US 2016/0279124 A1
(43) Pub. Date: Sep. 29, 2016

(54) Method for Treating Addiction

(71) Applicants: Dan Markel, Toluca Lake, CA (US); Brian Newton, Glendale, CA (US)

(72) Inventors: Dan Markel, Toluca Lake, CA (US); Brian Newton, Glendale, CA (US)

(21) Appl. No.: 15/082,565

(22) Filed: Mar. 28, 2016

Related U.S. Application Data
(60) Provisional application No. 62/139,287, filed on Mar. 27, 2015.

(51) Int. Cl. A61K 31/485

(52) U.S. Cl. CPC A61K 31/485

(57) ABSTRACT

The present invention provides a method of treating a patient suffering from addiction, having the steps of administering to the patient a controlled release composition comprising an opioid antagonist; and coaching the patient.

### CROSS-REFERENCE TO RELATED APPLICATIONS

The present application claims priority to U.S. Provisional Patent Application 62/139,287, filed Mar. 27, 2015, the entire contents of which is incorporated herein by reference.

### FIELD OF THE INVENTION

The present invention relates to the use of a long-lasting naltrexone implant combined with a unique life coaching program for treatment of addictions. (drugs, alcohol, food, gambling, other addictions)

### BACKGROUND OF THE INVENTION

23 million Americans suffer from addiction.¹ Average success rates for any of the existing treatments in the US and internationally are typically between 5 and 10%. It's estimated that the cost of opioid and alcohol addiction combined is nearly $800 billion annually.² There is a huge unmet need for a much more effective treatment of alcohol or opioid addiction. Even with a solution that can provide simply a doubling of the efficacy of current approaches the costs savings for health care alone would be enormous not to mention savings from the reduction in crime, accidents and lost wages. More importantly the benefits to society would also be immeasurable in terms of lives and families saved.

### PRIOR ART

The entire ecosystem of the alcohol recovery and drug addiction rehabilitation is fragmented, uneven and for the most part highly ineffective. Beginning with Alcoholics Anonymous: Even with its noble elements of community and service, it is still sadly failing for at least 90% of those individuals seeking to become and stay sober. The fragmentation is even more shocking with virtually no regulation and myriad of facilities run by ex addicts/alcoholics, all offering essentially the same psychosocial treatment that fails almost all of the time. The failure does not seem to be related to the sincere desire for the person to quit drinking or stop taking the opiate, but rather in the person's inability to overcome the intense physical cravings that heretofore have always been treated with “white knuckling” through the 6-12 months needed to allow proper healing of a diseased, addicted brain. Currently as outlined below there are no comprehensive, well managed, programs with professional medical oversight offering the combined medical and psychosocial treatment.

| Name | Type | Services | Cost | Success |
|---|---|---|---|---|
| AA, | 12-Step | Group | free | 5-10% |
| Al-Anon, etc | Programs | counseling | | |
| AAC, FRN, | 30 day | Inpatient | $28,000 to | ~20%³ |
| Schick Shadel, | rehab | 30 day | $60,000 per bed | |
| Passages, | centers | behavioral | per 30-day stay | |
| Cliffside, etc. | | therapy | | |
| Clinic and | Medical | Medical Detox | $1,000 to $5,000 | 8% for |
| office based | treatment | Naltrexone | depending on | opioid |
| medical | | treatment: pill, | extent of medical | use |
| treatment | | injectable and | treatment and | |
| | | implant | drugs prescribed | |
| The Coleman | Medical | Naltrexone pill, | $1K to $5K | N/A |
| Institute | treatment | injectable and 3 | depending | |
| | | month implants | on extent | |
| | | | of treatment | |
| Sober Life | Medical | Long-lasting | $28,000 per | N/A |
| USA | treatment | Naltrexone | procedure | |
| | | implant | | |

Nearly all current treatment modalities have the following in common: 1) they are all behavioral, 12-step/Alcoholics Anonymous based; 2) They all treat the disease of addiction and alcoholism as an acute condition as opposed to a chronic medical ailment such as cancer, diabetes, heart disease or Hepatitis, typically programs only last for 30-60 days in current treatment programs. 3) There is currently no company offering a comprehensive medical approach to the disease of addiction other than MyLife Recovery Centers, Inc. (MRLC).

### SUMMARY OF THE INVENTION

MyLife Recovery Centers, Inc. is uniquely positioned to be the US and global leader in the first comprehensive long term dual treatment of addiction and alcoholism. MyLife uses Medical Groups owned and operated by medical doctors to provide the medical treatment. MyLife has developed a solution that offers success rates of about 80%—eight times the average effectiveness of current treatments. Its unique approach is based upon a revolutionary combined medical and psychosocial approach to treatment beginning with a surgical insertion of a long-lasting Naltrexone implant. The drug Naltrexone has been approved by the FDA for treatment of alcohol and opioid addiction and blocks the opioid receptors in the brain, removing or greatly reducing physical cravings for alcohol or opiates. The implant, prescribed by MyLife doctors and compounded by pharmacies, releases a daily dose of Naltrexone over a period of 6 to 12 months removing the need for patient compliance required in a pill or injectable form. Implant recipients no longer have to “white knuckle it” through coaching or therapy and can, therefore, be more receptive to the benefits of life coaching and therapy. MyLife achieves these very high rates of success exactly because it combines the highly effective implant with its very powerful and long term (12-60 months) proprietary coaching and counseling—the first dual treatment of alcohol and opiate addiction. An example of the implant that can be used in the present invention is described in U.S. Pat. No. 6,203,813.

Difference Between Current Practices and this Invention

To improve the chances of successful recovery, a combination of medical and psychosocial treatment is necessary. To date, treatment programs worldwide have offered only a short term, behavioral based, psychosocial approach for the afflicted. MyLife Recovery Centers, Inc. (“MyLife”) provides a holistic, revolutionary, two-pronged approach to treatment tailoring it to each patient's unique needs. MyLife's proprietary process begins with the surgical insertion of a long-lasting naltrexone implant that eliminates, or dramatically reduces the overwhelming physical cravings experienced by ALL alcoholics and opiate addicts. Furthermore, this procedure eliminates the challenging issue of patient compliance. With the naltrexone implant the medication is released at daily therapeutic levels for 6 to 12 months. This simple outpatient and private thirty-minute procedure coupled with a customized life coaching program produces success rates of approximately 80%. Free from physical cravings patients are far more receptive and attentive to MyLife's customized and individualized therapy and coaching program.

Addiction Recovery Life Coaching

The unique knowledge and cross functional skills of the MyLife team enable the Company to provide a customizable and individualized life coaching and therapy program for each patient that greatly enhances the prospects for patients to continue a long term substance free lifestyle. MyLife considers this program to be an enormous differentiator from other recovery programs and a significant component of its competitive advantage.

Accordingly, the present invention provides a method of treating a patient suffering from addiction, comprising the steps of:

administering to the patient a controlled release composition comprising an opioid antagonist; and coaching the patient comprising

a) collecting patient history and current status;

b) outlining expectations of treatment results;

c) identifying and altering patient's addictive behavior;

d) identifying or developing a support system to facilitate patient recovery from addiction; and

e) conducting follow-up sessions with the patient to reassess a) to d) and track progress of treatment.

In one embodiment, step c) above comprises the step of identifying the cause for patient's addictive behavior selected from the group consisting of triggers, feelings, habits, influences, thought processes, and self-esteem issues.

In one embodiment, the method of the present invention is useful in treating a person having alcohol or opiate addiction. The controlled release implant releases effective daily dose of the opioid antagonist for at least three months to a8 months; preferably the controlled release implant releases an effective daily dose of the opioid antagonist over a period of 6 to 12 months. More preferably, the controlled release composition is administered parenterally as an implant to the patient.

In one embodiment of the invention, the method of the present invention further comprises detoxing the patient before administering to the patient the controlled release composition.

Examples of opioid antagonists that can be used in the method of the present invention are naltrexone, naloxone, buprenorphine, nalmefene, cyprodime, naltrindole, and norbinaltorphimine. More preferably, the opioid antagonist is naltrexone.

### DETAILED DESCRIPTION OF THE INVENTION

Description of Coaching Process

Each patient comes into MLRC with the same goal in mind “to stop practicing their addiction.” While each patient's goal is the same, the process involved to achieve that goal can take as many different avenues as there are patients, which is why the individualized, customized approach is most beneficial.

Unlike long term industry standards of treatment of “one size fits all” behavioral counseling/Life Coaching, at MyLife it is recognized that every patient is in a different stage of their disease and life circumstances. The insight into this approach was specifically ascertained by treating the patients first with the Naltrexone implant that greatly reduces or eliminates the deep cravings and actually allows the intake of the coaching/counseling. An addict or alcoholics brain in the early stages of sobriety is simply not equipped without the naltrexone to absorb the advice being given by the coach or counselor. Hence the incredibly high relapse rates of patient's right after they leave residential treatment centers.

MyLife's approach recognizes that some will progress rapidly, others more slowly. The longer a patient has used the addictive substance the higher chance of cognitive impairment, so the ability to process and the willingness of the patient to change addictive behaviors and addictive thinking plays a large role in the patient's success.

Frequent and Long term Coaching is a cornerstone of MyLife's Program.

Key differentiators for MyLife are duration and frequency. Face to face sessions are preferred but when not possible tele coaching can be an alternative. Often, phone contact between sessions is necessary for added support, especially early on in the process.

The Life Coach (LC) will be looking for signs or symptoms of secondary diagnosis and determine if patient needs referral for a psych evaluation and/or therapy in conjunction with coaching.

First Session
build a relationship through empathy, understanding, and identification
   patient needs to know and feel that the LC “gets it”
   rapport and trust must be established
discuss expectations
   LC expects
      participation—discuss cancelation policy
      100% honesty is imperative
      confidentiality for both is expected
      discuss limits of confidentiality pertaining to mandatory reporting laws
   patient expectations—what is patient hoping to get out of coaching
      manage any unrealistic expectations
set up coaching schedule
exchange contact information and preferred form of communication
   discuss if those at home are aware patient is in the MLRC program
   if family is unaware discuss ways to protect confidentiality
review any critical items on the life coaching packet forms patient filled out and address any immediate concerns

Address Detox

If it is a session prior to implant and patient still needs to detox:
what do they think their chances of a successful detox are
have they previously detoxed
   if so, what did they experience
      what was the most challenging part
      discuss strategies to get through it
      manage expectations—physical, emotional and social
      what are the roadblocks or hurdles/challenges
      what do they say to their family and friends

Help Patient Identify the Most Appropriate Detox Process (in Collaboration with Medical Staff)
is medication needed
if so, does patient have resistance to taking what is prescribed
are they capable of managing their own prescriptions or do they need support
is home detox possible or is medical detox necessary
   if home detox is possible
      what will that be like—is this likely to be successful
      who is at home to support
      is anyone else at home drinking or using currently
      is it a safe environment—no addictive substances available
      get rid of all addictive substances
         stash left in car, home, work, garage, etc. —make environment free of triggering substances paraphernalia
         is patient able to clean out and discard on his own
         if not, identify a safe person who would be able to assist in safely discarding

If Medical Detox is Necessary
how long do they need to detox (depends on substance)
identify appropriate facility
what support do they need to take care of things at home while in detox facility
make sure home is free of addictive substances upon return home (see above)

If Patient is Already Detoxed or when Detoxed:

Information Gathering:
during sessions the coach will learn what patient has tried in the past to get clean and sober
   what helped, what didn't
discuss current status and or belief systems with their:
   family life, employment, relationships, spirituality, hobbies, recreational choices, social life, finances, etc.
identify and explore current feelings—are they based in the past or present
   note where the patient is emotionally charged
current habits
times of day that are dangerous or triggering
who are their most influential friends—in positive and negative ways

With this Information the Coach Will Partner with the Patient to:
manage expectations—this process is going to feel uncomfortable
explore solutions to learn to process feelings in a healthy way
identify and establish healthy coping skills
identify patients unhealthy patterns and relapse behaviors
discuss tools and strategies to begin to alter their addictive patterns
create an action plan
   immediate action when “in trouble” (what to do in the moment of having first thoughts)
   actions to support long term recovery
support system is critical
   identify if a healthy current support system exists
   begin to develop if none exists
   continue to develop—what this looks like will change; as the patient gets healthier, their support needs change

Ongoing

During sessions there will be ongoing discussion regarding the following:
identifying triggers, feelings, potential trouble spots, habits, influences, thought processes, self-esteem issues, etc.
creating tools and responses that support healthy recovery
discussing since last session:
   what worked, where were the successes
   what didn't
   what needs to be altered to be more effective

If Patient Relapses or is Having Thoughts of Using, Explore the Following:
what are the circumstances or situation
time of day
people involved—often family or friends closest to patient
place or environment
habits
self-sabotaging behaviors
what can they do differently

Explore Triggering Feelings:
isolation
grief
loneliness
hunger
anger
tired
frustrated
hopeless
fear of success
fear of failure
stress

Life Coach then Supports Patient to:
process feelings
develop new strategies
create new approaches
challenge old thinking
   pros and cons
   is the thought true
   is it past or present
   does it really make you feel better or is it just temporary
try new ways of working through and overcoming situations and feelings that are triggering
explore what coping skills worked in the past
   what new coping skills are needed to achieve new found contentment in sobriety.

Key Tools & Components

Foundational Coaching Principles/Techniques—

specific coaching basics that facilitate change addiction recovery in participants. The following lists the important coaching basics.

Positive Psychology

The scientific study of human flourishing, and an applied approach to optimal functioning. It has also been defined as the study of the strengths and virtues that enable individuals, communities and organizations to thrive.

Neurolinguistics Programming

Neurolinguistics Programming (NPL) focuses on the neurolinguistics structures within the individual. NLP encompasses the three most influential components involved in producing human experience: neurology, language and programming. The neurological system regulates how our bodies function, language determines how we interface and communicate with other people and our programming determines the kinds of models of the world we create. Neuro-Linguistic Programming describes the fundamental dynamics between mind (neuro) and language (linguistic) and how their interplay affects our body and behavior (programming).

Strategic Intervention (SI)

Strategic Intervention focuses on the interactivity in relationships and the context of the person in their social group and in their life. Belief in the importance of understanding a person's relationships, stage in life, cultural influences, and beliefs before they can be transformed by means of a skillfully planned strategy. In SI a coach might give a client a strategy to carry out at home that will solve problems by transforming multiple relationships and therefore creating new opportunities.
Daily Solution Integration—worksheets, templates, and workflows directly related to foundational coaching priciples/techniques. These include key coaching questions, key action steps, key guided visualizations/imagery/meditation/exercises, as well as complementary coaching techniques. The key action steps are the following:

Key Steps
1. Establish relationship and trust
2. Ask questions and demonstrate curiosity
3. Listen and demonstrate intuition
4. Solicit feedback and look for awareness
5. Suggestions and opportunities to simplify
6. Establish goals and develop action plans
7. Define accountability and track accomplishments
Key Coaching Tools & Techniques—valuable coaching tools that further enhance addiction recovery outcomes.

Program strategically integrates the tools that have been proven to work, including positive psychology, neuro-linguistic programming, cognitive therapy, strategic intervention, guided imagery and visualization, meditation, self-actualization tools, positive self-talk, correction of negative self-perceptions, the law of attraction, Gestalt therapy, cognitive therapy and countless other techniques and teachings.
Guided Visualization/Imagery/Mediation/Exercises Training—Training on how to use guided coaching techniques to facilitate breakthroughs, including a review of the leading methods, and what and where to use. The leading methods include the following:
Positive psychology
Neuro-linguistic programming
Cognitive therapy
Strategic intervention
Guided imagery and visualization
Meditation,
Self-actualization tools
Positive self-talk
Projection of negative self-perceptions
the law of attraction
Gestalt therapy
Cognitive therapy and countless other techniques and teachings.
Early Sobriety Matrix—Documentation covering greatest early sobriety challenges clients face, and coaching tools and techniques to help them overcome these challenges.
Peer Coaching Model—Training on how to leverage the power of peer coaching (one addict/alcoholic to another).
Video & Audio Coaching—Multimedia training on core components of the coaching program, which at a minimum will include the most important tools and techniques used in coaching. This may include practice coaching calls demonstrating techniques
Detailed Mind Maps—Mind maps outlining coaching practices, techniques, and tools that link to the corresponding processes so coaches are able to quickly work with clients identifying challenges and resolutions.
The Mind maps will identify from client the following to facilitate the best possible results:

values>thoughts>beliefs>feelings and emotions>actions>results
Client Tracking Framework—Documentation process for coaches to follow to track ongoing challenges, successes, opportunities, goals, progress, notes, etc.

### Additional Embodiments of the Invention

### Intensive Outpatient Program

To further enhance the likelihood of successful recovery outcomes, MyLife is developing an Intensive Outpatient Program (IOP). The program is a significant differentiator as it extends the service offering providing additional tools and resources to assist patients with their recovery. In MyLife Recovery Centers IOP treatment program, clients learn about the disease of addiction and acquire the tools to help get them on the road to recovery. At MyLife Recovery Centers, counselors do individual treatment planning as well as group and individual counseling. MyLife expects that this program will become an important offering to attract and retain customers/patients.

The purpose of the IOP is to empower patients to develop healthy and responsible functioning through productive and chemical-free living. The program provides the following:
Structured, supportive therapeutic environment helpful to introducing and continuing recovery behaviors
Chemical dependency and co-occurring disorder counseling by trained and experienced clinicians
Comprehensive assessments to fully disclose chemical use issues and other psychiatric disorders
Individual, group, and family therapy on a regular basis with consideration to the level of need
Spiritual counseling and emphasize the need for a higher power in recovery
Pharmacological services as needed

### Footnotes

1. SAMHSA
2. http://www.alcohol-information.com & http://www.drug-addiction-support.org
3. Per CEO of multi-location residential recovery program

What is claimed:

1. A method of treating a patient suffering from addiction, comprising the steps of:
administering to the patient a controlled release composition comprising an opioid antagonist;
and coaching the patient comprising
a) collecting patient history and current status;
b) outlining expectations of treatment results;
c) identifying and altering patient's addictive behavior;
d) identifying or developing a support system to facilitate patient recovery from addiction; and
e) conducting follow-up sessions with the patient to reassess a) to d) and track progress of treatment.

2. The method of claim 1, wherein the addiction is alcohol or opiate addiction.

3. The method of claim 1, wherein the controlled release implant releases an effective daily dose of the opioid antagonist over a period of 6 to 12 months.

4. The method of claim 1, wherein the controlled release composition is administered parenterally as an implant to the patient.

5. The method of claim 1, further comprising detoxing the patient before administering to the patient the controlled release composition.

6. The method of claim 1, wherein the opioid antagonist is selected from the group consisting of naltrexone, naloxone, buprenorphine, nalmefene, cyprodime, naltrindole, and norbinaltorphimine.

7. The method of claim 1, wherein the opioid antagonist is naltrexone.

8. The method of claim 1, wherein step c) comprises the step of identifying the cause for patient's addictive behavior selected from the group consisting of triggers, feelings, habits, influences, thought processes, and self-esteem issues.
